(19) United States
(12) Patent Application Publication
(10) Pub. No.: US 2013/0126429 A1
(43) Pub. Date: May 23, 2013

(54) AMINE SULFIDE SCAVENGERS, METHODS OF USING, AND METHODS OF MAKING

(71) Applicants: Ping Lue, Trevose, PA (US); Gregory Kaplan, Richboro, PA (US)

(72) Inventors: Ping Lue, Trevose, PA (US); Gregory Kaplan, Richboro, PA (US)

(73) Assignee: GENERAL ELECTRIC COMPANY, Schenectady, NY (US)

(21) Appl. No.: 13/303,331

(22) Filed: Nov. 23, 2011

(51) Int. Cl. B01D 15/04; C07D 413/12; B01D 53/14; C07D 417/12; C07C 217/08; C07D 401/12; C07D 403/12

(52) U.S. Cl. 210/638; 546/186; 544/86; 544/357; 544/60; 564/508; 564/504; 95/235; 95/237

(57) ABSTRACT

Methods for making sulfide scavenging compositions are provided. The compositions are secondary amine-formaldehyde adduct (SAFA) scavengers comprising less than about 40 wt % N-methyl secondary amines therein. The methods include reacting secondary amines with formaldehyde. Methods for removing sulfides from fluid streams are also provided. The methods include adding secondary amine-formaldehyde adduct (SAFA) scavengers to fluid streams. SAFA scavengers comprising less than about 40 wt % N-methyl secondary amines of the total weight of SAFA scavengers are also disclosed.

### FIELD OF INVENTION

The invention pertains to methods and chemical compositions for reacting with sulfides, and more particularly, for scavenging sulfides from water and hydrocarbon streams.

### BACKGROUND OF THE INVENTION

Hydrogen sulfide, or H₂S, is a clear, toxic gas with a foul odor. It is also highly flammable. The Environmental Protection Agency and other regulatory agencies worldwide strictly control the release of H₂S into the environment. H₂S is often present in well water, waste water and other aqueous systems. H₂S is often present in crude oil and natural gas reserves and must be removed before making commercial use of such reserves. The H₂S concentration in these reserves prior to treatment typically varies with location and is usually higher in natural gas than in crude oil reserves. In natural gas reserves, for example, H₂S may vary from less than 100 ppm to 3000 ppm. Permitted H₂S levels will also vary by location. The U.S. limits H₂S in natural gas pipelines to 4 ppm per 100 standard cubic feet (0.3 gr/100 scf).

Generally, hydrocarbon streams are treated to remove sulfides, including organic sulfides, mercaptans, thiols, COS, and H₂S, by using chemicals that will react with the sulfides. These chemicals are called scavengers, or sweetening agents. These chemical scavengers include adducts produced through the reaction of secondary amines and formaldehydes. These secondary amine-formaldehyde adduct (SAFA) scavengers include triazines, oxazolidines, Schiff bases, diamines, methyol adducts, and methylene bridge materials.

Most hydrocarbon reserves are treated continuously near the wellhead, though treating hydrocarbons in a batch or similar application elsewhere is not uncommon. Continuous treatment installations near the wellhead inject scavengers, including SAFA scavengers, directly into the hydrocarbon pipeline. The injection system typically includes a chemical injection pump and piping tees or atomization nozzles to introduce the scavengers into the pipeline. The amount of scavengers required will vary depending on a variety of factors including, the type of scavengers used, the amount of H₂S in the well, permissible H₂S limits, and the well flow rate. Thus, the amount of scavengers added to treat a hydrocarbon pipeline typically ranges from approximately 10 ppm to about 100,000 ppm by volume of the hydrocarbon stream. A length of the pipeline is provided to allow for contact between the scavenger and the sulfide.

### BRIEF DESCRIPTION OF THE INVENTION

It was surprisingly discovered that some secondary amine-formaldehyde adducts, N-methyl secondary amines, are inert with H₂S. These deleterious N-methyl secondary amines have a methyl group and lack an ether or polyether group, making them inert with respect to H₂S. N-methyl secondary amines are often present in SAFA scavengers. N-methyl secondary amines may have the structure as set forth in formula I or II:

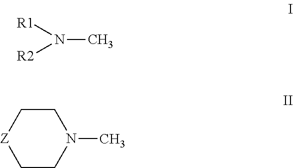

where R₁, and R₂ may be the same or different alkyls, hydroxyl-substituted alkyls, and alkoxy-substituted alkyls of 1 to 20 carbon atoms; the alkyl groups may be straight or branched alkyl groups, including, but not limited to, methyl, ethyl, propyl, butyl, hydroxylethyl, and methoxypropyl; and the cycloalkyl ring has an atom, Z, selected from the group consisting of carbon, oxygen, nitrogen, including NH (piperazine), piperidine, morpholine, and sulfur.

N-methyl secondary amines include cycloalkylmethylamines, dialkylmethylamines, and tertiary amines. Examples of N-methyl secondary amines include, but are not limited to, diethylmethylamine, dipropylmethylamine, dibutylmethylamine, N-methyl piperazine, N-methyl piperidine, N-methyl morpholine, and N,N-dimethylmethanamine.

It was also surprisingly discovered that the production of N-methyl secondary amines can be controlled by controlling the molar ratios of the reagents. Accordingly, the first embodiment discloses a method for making secondary amine-formaldehyde adduct (SAFA) scavengers, wherein the yield of N-methyl secondary amines is less than about 40 wt % of the total (SAFA) scavengers produced. The method comprises providing a reaction vessel charged with formaldehyde and reacting the formaldehyde with at least one secondary amine. In another embodiment, at least one of the secondary amines used includes a dialkylamine, including, but not limited to, di-n-propylamine, di-n-butylamine or di-n-pentylamine. In another embodiment, the molar ratio of secondary amines to formaldehyde ranges from about 1.0 to 1.95 to about 1.0 to 1.0. In another embodiment, the molar ratio of secondary amines to formaldehyde ranges from about 1.0 to 1.75 to about 1.0 to 1.25. In yet another embodiment, the molar ratio of secondary amines to formaldehyde ranges from about 1.0 to 1.60 to about 1.0 to 1.40.

In other embodiments, the SAFA scavengers produced may have the structure as set forth in formula III, IV, or V:

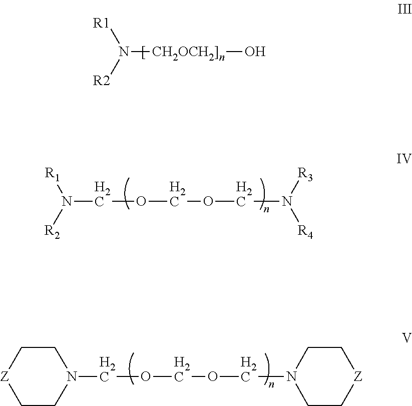

where n may be 1 to 100; where R₁, R₂, R₃, and R₄ may be the same or different alkyls, hydroxyl-substituted alkyls, and alkoxy-substituted alkyls of 1 to 20 carbon atoms; the alkyl groups may be straight or branched alkyl groups, including, but not limited to, methyl, ethyl, propyl, butyl, hydroxylethyl, and methoxypropyl; and the cycloalkyl ring has an atom, Z, selected from the group consisting of carbon, oxygen, nitrogen, including NH (piperazine), piperidine, morpholine, and sulfur.

In yet other embodiments, the SAFA scavengers produced may have the structure as set forth in formula VI or VII:

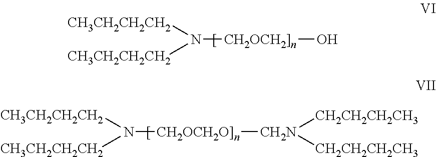

where n may be 1 to 100.

It was also surprisingly discovered that N-methyl secondary amines have a lower boiling point than secondary amine-formaldehyde adducts suitable for sulfide scavenging. The boiling point at 760 mmHg of many N-methyl secondary amines, including dibutylmethylamine, ranges from about 160 to about 170° C. Accordingly, another embodiment discloses a method for reducing N-methyl secondary amines from the SAFA scavengers produced through distillation.

In yet another embodiment, a method for making SAFA scavengers is disclosed, wherein the yield of N-methyl secondary amines is about 20 wt %. In yet another embodiment, the N-methyl secondary amines comprise dibutylmethylamine.

Another embodiment discloses a method for reducing sulfides from fluid streams is disclosed wherein the secondary amine-formaldehyde adduct (SAFA) scavengers used have reduced levels of N-methyl secondary amines. The method comprises providing a fluid stream and contacting the sulfides in the fluid stream with SAFA scavengers. The N-methyl secondary amines comprise less than about 40 wt % of the total weight of the SAFA scavengers. The sulfides reduced include organic sulfides, mercaptans, thiols, COS, and H₂S.

In another embodiment, the fluid stream is a hydrocarbon stream. In another embodiment, the fluid stream is an aqueous stream.

In another embodiment, the SAFA scavengers comprise less than about 20 wt % N-methyl secondary amines. In yet another embodiment, the SAFA scavengers comprise less than about 5 wt % N-methyl secondary amines. In another embodiment, the N-methyl secondary amines are dibutylmethylamine.

In another method, SAFA scavengers having less than 40 wt % N-methyl secondary amines are added to a fluid stream in an amount ranging from about 10 to about 100,000 ppm by volume of the fluid stream. In another method, SAFA scavengers are added to a fluid stream in an amount ranging from about 100 to about 50,000 ppm by volume of the fluid stream. In yet another method, SAFA scavengers are added to a fluid stream in an amount ranging from about 600 to about 3,000 ppm by volume of the fluid stream.

In another embodiment, secondary amine-formaldehyde adduct (SAFA) scavengers are disclosed comprising less than about 40 wt % N-methyl secondary amines. In yet other embodiments, the SAFA scavengers may have the structure as set forth in formula VI or VII:

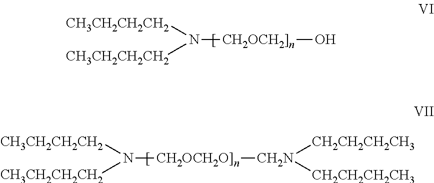

where n may be 1 to 100.

In another embodiment, the SAFA scavengers comprise less than about 20 wt % N-methyl secondary amines. In yet another embodiment, the SAFA scavengers comprise less than about 5 wt % N-methyl secondary amines.

### DETAILED DESCRIPTION OF EXEMPLARY EMBODIMENTS

Secondary amine-formaldehyde adducts suitable for sulfide scavenging include triazines, oxazolidines, Schiff bases, diamines, methyol adducts, and methylene bridge materials. Typically they are made by reacting a secondary amine with formaldehyde or paraformaldehyde. Suitable secondary amines include, but are not limited to, dialkylamines, dimethylamine, diethylamine, dipropylamine, dipentylamine, diethanolamine, morpholine, piperazine, and piperidine. These secondary amines have the structure as set forth in formula VIII or IX:

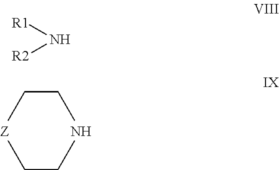

where R₁, and R₂ may be the same or different alkyls, hydroxyl-substituted alkyls, and alkoxy-substituted alkyls of 1 to 20 carbon atoms; the alkyl groups may be straight or branched alkyl groups, including, but not limited to, methyl, ethyl, propyl, butyl, hydroxylethyl, and methoxypropyl; and the cycloalkyl ring has an atom, Z, selected from the group consisting of carbon, oxygen, nitrogen, including NH (piperazine), piperidine, morpholine, and sulfur.

When reacted with formaldehyde, the above secondary amines form secondary amine-formaldehyde adduct (SAFA) scavengers having the structure as set forth in formula III, IV, or V:

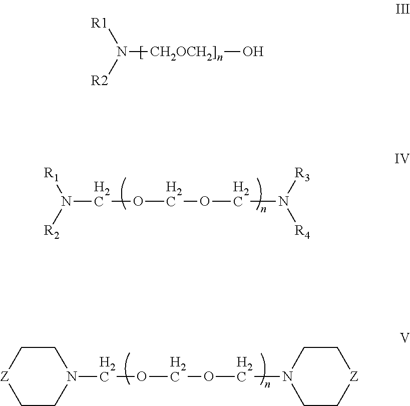

where n may be 1 to 100; where R₁, R₂, R₃, and R₄ may be the same or different alkyls, hydroxyl-substituted alkyls, and alkoxy-substituted alkyls of 1 to 20 carbon atoms; the alkyl groups may be straight or branched alkyl groups, including, but not limited to, methyl, ethyl, propyl, butyl, hydroxylethyl, and methoxypropyl; and the cycloalkyl ring has an atom, Z, selected from the group consisting of carbon, oxygen, nitrogen, including NH (piperazine), piperidine, morpholine, and sulfur. Alternative ranges for n include 1 to 20; 1 to 10; or 1 to 4.

It was surprisingly discovered that some secondary amine-formaldehyde adducts, N-methyl secondary amines, are inert with H₂S. These deleterious N-methyl secondary amines have a methyl group and lack an ether or polyether group, making them inert with respect to H₂S. N-methyl secondary amines are often present in SAFA scavengers. N-methyl secondary amines may have the structure as set forth in formula I or II:

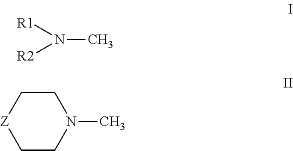

where R₁, and R₂ may be the same or different alkyls, hydroxyl-substituted alkyls, and alkoxy-substituted alkyls of 1 to 20 carbon atoms; the alkyl groups may be straight or branched alkyl groups, including, but not limited to, methyl, ethyl, propyl, butyl, hydroxylethyl, and methoxypropyl; and the cycloalkyl ring has an atom, Z, selected from the group consisting of carbon, oxygen, nitrogen, including NH (piperazine), piperidine, morpholine, and sulfur.

N-methyl secondary amines include cycloalkylmethylamines, dialkylmethylamines, and tertiary amines. Examples of N-methyl secondary amines include, but are not limited to, diethyl methylamine, dipropylmethylamine, dibutylmethylamine, N-methyl piperazine, N-methyl piperidine, N-methyl morpholine, and N,N-dimethylmethanamine.

Other effective SAFA scavengers are products of di-n-butylamine and formaldehyde. The most effective scavenging adducts of di-n-butylamine and formaldehyde have the structure as set forth in VI or VII:

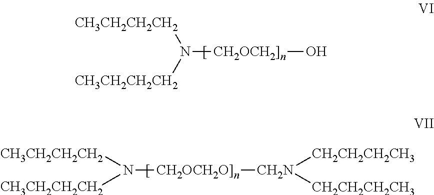

where n may be 1 to 100. Alternative ranges for n include 1 to 20; 1 to 10; or 1 to 4.

The undesired byproduct of the di-n-butylamine and formaldehyde reaction is dibutylmethylamine (DBMA). This byproduct is also known methyl-dibutylamine, N-methyl-di-n-butylamine, or N-butyl-N-methylbutan-1-amine. As with other types of deleterious N-methyl secondary amines, DBMA has a methyl group and lacks an ether or polyether group, making it inert with respect to H₂S. DBMA has the structure and formula X:

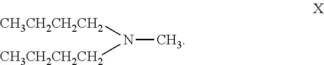

DBMA is not only inert with respect to H₂S; it flammable. In addition, DBMA is soluble in hydrocarbon and thus can negatively affect downstream hydrocarbon applications. Negative effects include increasing the nitrogen content as well as increasing the likelihood of corrosion and fouling of processing equipment. Concentrations of DBMA in many H₂S scavengers, however, may be as high as 55 wt % of the total weight of the H₂S scavengers. High DBMA concentrations in these scavengers not only have the negative impacts mentioned above but also increase the volume of scavengers required, increasing treatment costs. It also lowers the scavenger flash point, resulting in increased storage and shipping costs. Thus, di-n-butylamine-formaldehyde adducts with low DBMA concentrations are preferred in H₂S scavenging applications.

It was also surprisingly discovered that the production of N-methyl secondary amines can be controlled by controlling the molar ratios of the reagents. Accordingly, the first embodiment discloses a method for making secondary amine-formaldehyde adduct (SAFA) scavengers, wherein the yield of N-methyl secondary amines is less than about 40 wt % of the total (SAFA) scavengers produced. The method comprises providing a reaction vessel charged with formaldehyde and reacting the formaldehyde with at least one secondary amine. The secondary amine-formaldehyde adducts produced include, but are not limited to triazines, oxazolidines, Schiff bases, diamines, methyol adducts, and methylene bridge materials. In another embodiment at least one of the two secondary amines used includes a dialkylamine, including, but not limited to, di-n-propylamine, di-n-butylamine or di-n-pentylamine. In another embodiment at least one of the secondary amines used includes a dialkylamine, including, but not limited to, di-n-propylamine, di-n-butylamine or di-n-pentylamine. In another embodiment, the molar ratio of secondary amines to formaldehyde ranges from about 1.0 to 1.95 to about 1.0 to 1.0. In another embodiment, the molar ratio of secondary amines to formaldehyde ranges from about 1.0 to 1.75 to about 1.0 to 1.25. In yet another embodiment, the molar ratio of secondary amines to formaldehyde ranges from about 1.0 to 1.60 to about 1.0 to 1.40.

In other embodiments, the SAFA scavengers produced may have the structure as set forth in formula III, IV, or V:

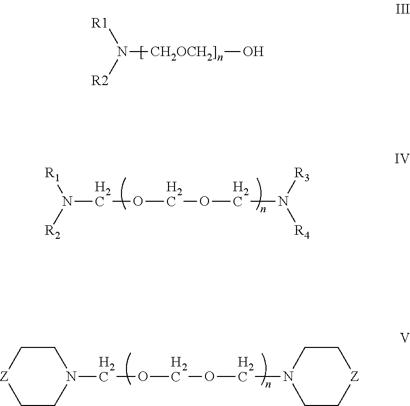

where n may be 1 to 100; where R₁, R₂, R₃, and R₄ may be the same or different alkyls, hydroxyl-substituted alkyls, and alkoxy-substituted alkyls of 1 to 20 carbon atoms; the alkyl groups may be straight or branched alkyl groups, including, but not limited to, methyl, ethyl, propyl, butyl, hydroxylethyl, and methoxypropyl; and the cycloalkyl ring has an atom, Z, selected from the group consisting of carbon, oxygen, nitrogen, including NH (piperazine), piperidine, morpholine, and sulfur. Alternative ranges for n include 1 to 20; 1 to 10; or 1 to 4.

In yet other embodiments, the SAFA scavengers produced may have the structure as set forth in formula VI or VII:

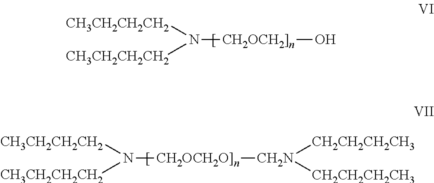

where n may be 1 to 100. Alternative ranges for n include 1 to 20; 1 to 10; or 1 to 4.

In yet another embodiment, a method for making SAFA scavengers is disclosed, wherein the yield of N-methyl secondary amines is about 20 wt %. In yet another embodiment, the N-methyl secondary amines comprise dibutylmethylamine.

It was also surprisingly discovered that N-methyl secondary amines have a lower boiling point than secondary amine-formaldehyde adducts suitable for sulfide scavenging. The boiling point at 760 mmHg of many N-methyl secondary amines, including dibutylmethylamine, ranges from about 160 to about 170° C. Accordingly, another embodiment discloses a method for reducing N-methyl secondary amines from SAFA scavengers produced in the prior methods through distillation. SAFA scavengers used in this embodiment include, but are not limited to, triazines, oxazolidines, Schiff bases, diamines, methyol adducts, and methylene bridge materials. Any water present in SAFA scavengers may be separated using a water separator or membrane, or any other method known to those of ordinary skill in the art. The SAFA scavengers are then distilled using suitable distillation processes known to those of ordinary skill in the art, including but not limited to, continuous, single stage, fractional, batch distillation or vacuum distillation. A distillation apparatus is charged with SAFA scavengers. The SAFA scavengers are distilled producing a vapor stream comprising N-methyl secondary amines as distillate and a liquid stream as bottoms. The vapor stream is removed. The bottoms are retained as purified SAFA scavengers comprising less than about 40 wt % N-methyl secondary amines of the total bottoms weight.

Another embodiment discloses a method for removing sulfides, including organic sulfides, mercaptans, thiols, COS, and H₂S, from hydrocarbon streams. The method comprises providing a liquid, gaseous, or mixed-phase hydrocarbon stream and contacting the sulfides in the hydrocarbon stream with secondary amine-formaldehyde adducts. These secondary amine-formaldehyde adducts comprise less than about 40 wt % N-methyl secondary amines. In another embodiment, the secondary amine-formaldehyde adducts comprise less than about 20 wt % N-methyl secondary amines. In yet another embodiment, the secondary amine-formaldehyde adducts comprise less than about 5 wt % N-methyl secondary amines. In another embodiment, the N-methyl secondary amines comprise dibutylmethylamine.

Optionally, the SAFA scavengers are vacuum distilled. The pressure is maintained at from about 0.1 to about 760 mm Hg. In another embodiment, the temperature is maintained at from about 45 to about 170° C. In yet another embodiment, the pressure is maintained at from about 10 to about 15 mm Hg, and the temperature is maintained at from about 50 to about 80° C. Distillation stops after most of the N-methyl secondary amines are distilled off. The bottom fraction is retained and contains purified SAFA scavengers.

Another embodiment discloses a method for reducing sulfides from fluid streams wherein the secondary amine-formaldehyde adduct (SAFA) scavengers used have reduced levels of N-methyl secondary amines. The method comprises providing a fluid stream and contacting the sulfides in the fluid stream with SAFA scavengers. The N-methyl secondary amines comprise less than about 40 wt % of the total weight of the SAFA scavengers. Suitable SAFA scavengers include but are not limited to, triazines, oxazolidines, Schiff bases, diamines, methyol adducts, and methylene bridge materials. The sulfides reduced include organic sulfides, mercaptans, thiols, COS, and H₂S.

In another embodiment, the fluid stream is a hydrocarbon stream. In another embodiment, the fluid stream is an aqueous stream.

In another embodiment, the SAFA scavengers used to scavenge sulfides comprise less than about 20 wt % N-methyl secondary amines. In yet another embodiment, the SAFA scavengers comprise less than about 5 wt % N-methyl secondary amines. In another embodiment, the N-methyl secondary amines are dibutylmethylamine.

In other embodiments, a method for reducing sulfides from fluid streams is disclosed wherein the SAFA scavengers used comprise dibutylamine-formaldehyde adduct scavengers. These scavengers have the structure as set forth in formula III or IV:

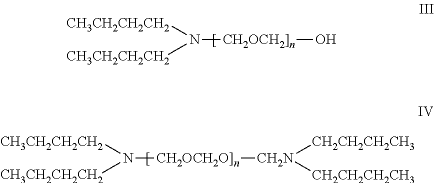

where n may be 1 to 100. Alternative ranges for n include 1 to 20; 1 to 10; or 1 to 4.

The amount of secondary amine-formaldehyde adducts added will depend on the application and amount of sulfide scavenging required. In another method, SAFA scavengers having less than 40 wt % N-methyl secondary amines are added to a fluid stream in an amount ranging from about 10 to about 100,000 ppm by volume of the fluid stream. In another method, SAFA scavengers are added to a fluid stream in an amount ranging from about 100 to about 50,000 ppm by volume of the fluid stream. In yet another method, SAFA scavengers are added to a fluid stream in an amount ranging from about 600 to about 3,000 ppm by volume of the fluid stream.

In other aspects, the fluid stream treated can comprise a fluid hydrocarbon stream or an aqueous fluid stream. These fluid streams may, for example, comprise gas/liquid mixtures from oilfield processes, pipelines, tanks, tankers, refineries, and chemical plants. Additionally the fluid stream may comprise farm discharge, city water, etc. Other additional fluid streams include water, waste water and process water containing H₂S.

In another embodiment, secondary amine-formaldehyde adduct (SAFA) scavengers are disclosed comprising less than about 40 wt % N-methyl secondary amines. In yet other embodiments, the SAFA scavengers may have the structure as set forth in formula VI or VII:

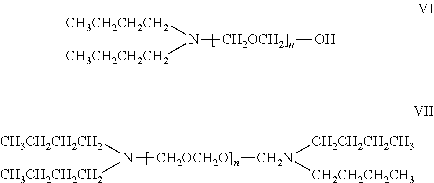

where n may be 1 to 100. Alternative ranges for n include 1 to 20; 1 to 10; or 1 to 4.

In another embodiment, the SAFA scavengers comprise less than about 20 wt % N-methyl secondary amines. In yet another embodiment, the SAFA scavengers comprise less than about 5 wt % N-methyl secondary amines.

### EXAMPLES

Comparative Example 1. Secondary amine-formaldehyde adducts were made using a molar ratio of di-n-butylamine to formaldehyde of 1.0 to 2.5. The reaction vessel was a four-necked, 300 mL, round-bottom flask equipped with a mechanical stirrer, a thermocouple with a controller, and a reflux condenser. 1.67 moles of formaldehyde (50.19 grams of paraformaldehyde) were placed in the flask under nitrogen. 0.67 moles (86.41 grams) di-n-butylamine were added to the flask over 1.5 hours. The reaction mixture was heated to 85° C. and held for 2 hours. The mixture temperature was then increased to 95° C. and held for an additional 2 hours. The mixture was then cooled and the organic layer separated from the aqueous layer. The organic layer was washed with water and the aqueous layer was again separated. The organic layer was then filtered and yielded 77 grams of clear, colorless secondary amine-formaldehyde adducts. GC-MS and nucleus magnetic resonance analysis showed the secondary amine-formaldehyde adducts comprised 86% dibutylmethylamine (DBMA) and 14% di-n-butylamine-formaldehyde adducts.

Comparative Example 2. Secondary amine-formaldehyde adducts were made using a molar ratio of di-n-butylamine to formaldehyde of 1.0 to 2.0. The reaction vessel in Comparative Example 1 was used. 1.5 moles of formaldehyde (45.03 grams of paraformaldehyde) were placed in the flask under nitrogen. 0.75 moles (96.93 grams) di-n-butylamine were added to the flask over 1.5 hours. The same mixing, washing and separation techniques in Comparative Example 1 were applied to yield 114 grams of clear, colorless secondary amine-formaldehyde adducts. GC-MS and nucleus magnetic resonance analysis showed the secondary amine-formaldehyde adducts comprised 45% dibutylmethylamine (DBMA) and 55% di-n-butylamine-formaldehyde adducts.

Example 1. Secondary amine-formaldehyde adducts were made using a molar ratio of di-n-butylamine to formaldehyde of 1.0 to 1.75. The reaction vessel in Comparative Example 1 was used. 1.56 moles of formaldehyde (46.74 grams of paraformaldehyde) were placed in the flask under nitrogen. 0.89 moles (115.19 grams) di-n-butylamine were added to the flask over 1.5 hours. The same mixing, washing and separation techniques in Comparative Example 1 were applied to yield 120 grams of clear, colorless secondary amine-formaldehyde adducts. GC-MS and nucleus magnetic resonance analysis showed secondary amine-formaldehyde comprised 36% dibutylmethylamine (DBMA) and 64% di-n-butylamine-formaldehyde adducts.

Example 2. Secondary amine-formaldehyde adducts were made using a molar ratio of di-n-butylamine to formaldehyde of 1.0 to 1. 50. The reaction vessel in Comparative Example 1 was used. 1.34 moles of formaldehyde (40.20 grams of paraformaldehyde) were placed in the flask under nitrogen. 0.89 moles (115.20 grams) di-n-butylamine were added to the flask over 1.5 hours. The same mixing, washing and separation techniques in Comparative Example 1 were applied to yield 131.4 grams of clear, colorless secondary amine-formaldehyde adducts. GC-MS and nucleus magnetic resonance analysis showed the secondary amine-formaldehyde adducts comprised 20% dibutylmethylamine (DBMA) and 80% di-n-butylamine-formaldehyde adducts.

This written description uses examples to disclose the invention, including the best mode, and also to enable any person skilled in the art to practice the invention, including making and using any devices or systems and performing any incorporated processes. These examples are merely illustrative and do not limit the invention in any manner. For example, while specific reaction conditions, such as temperature and time are disclosed, the desired reaction may also occur under alternate reaction conditions. The patentable scope of the invention is defined by the claims, and may include other examples that occur to those skilled in the art. These other examples are intended to be within the scope of the claims if they have structural elements that do not differ from the literal language of the claims, or if they include equivalent structural elements with insubstantial differences from the literal language of the claims.

What is claimed is:

1. A method for making secondary amine-formaldehyde adduct (SAFA) scavengers, wherein the yield of N-methyl secondary amines is less than about 40 wt %, comprising:
(a) providing a reaction vessel charged with formaldehyde or paraformaldehyde; and
(b) reacting said formaldehyde or paraformaldehyde with at least one secondary amine.

2. The method of claim 1, wherein at least one of said secondary amines includes a dialkylamine.

3. The method of claim 1, wherein at least one of said secondary amines includes di-n-propylamine, di-n-butylamine or di-n-pentylamine.

4. The method of claim 1, wherein at least one of said secondary amines includes di-n-butylamine.

5. The method of claim 1, wherein a molar ratio of said secondary amines to said formaldehyde or paraformaldehyde ranges from about 1.0 to 1.95 to about 1.0 to 1.0.

6. The method of claim 1, wherein a molar ratio said secondary amines to said formaldehyde or paraformaldehyde ranges from about 1.0 to 1.75 to about 1.0 to 1.25.

7. The method of claim 1, wherein a molar ratio said secondary amines to said formaldehyde or paraformaldehyde ranges from about 1.0 to 1.60 to about 1.0 to 1.40.

8. The method of claim 1, wherein said secondary amine-formaldehyde adduct (SAFA) scavengers comprise SAFA scavengers with the formula: 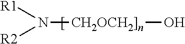
where n may be 1 to 100; R₁ and R₂ are the same or different alkyls, hydroxyl-substituted alkyls, and alkoxy-substituted alkyls of 1 to 20 carbon atoms; and said alkyls are straight or branched alkyl groups, including, but not limited to, methyl, ethyl, propyl, butyl, hydroxylethyl, and methoxypropyl.

9. The method of claim 1, wherein said secondary amine-formaldehyde adduct (SAFA) scavengers comprise SAFA scavengers with the formula: 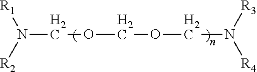
where n may be 1 to 100; R₁, R₂, R₃, and R₄ are the same or different alkyls, hydroxyl-substituted alkyls, and alkoxy-substituted alkyls of 1 to 20 carbon atoms; and said alkyls are straight or branched alkyl groups, including, but not limited to, methyl, ethyl, propyl, butyl, hydroxylethyl, and methoxypropyl.

10. The method of claim 1, wherein said secondary amine-formaldehyde adduct (SAFA) scavengers comprise SAFA scavengers with the formula: 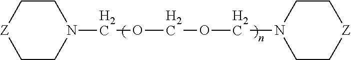
where n may be 1 to 100; and a cycloalkyl ring has an atom, Z, selected from the group consisting of carbon, oxygen, nitrogen, including NH (piperazine), piperidine, morpholine, and sulfur.

11. The method of claim 1, wherein said secondary amine-formaldehyde adduct (SAFA) scavengers comprise SAFA scavengers with the formula: 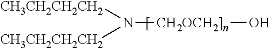
where n may be 1 to 100.

12. The method of claim 1, wherein said secondary amine-formaldehyde adduct (SAFA) scavengers comprise SAFA scavengers with the formula: 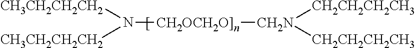
where n may be 1 to 100.

13. The method of claim 1, further comprising distilling said secondary amine-formaldehyde adduct (SAFA) scavengers.

14. The method of claim 1, wherein said N-methyl secondary amines comprise dibutylmethylamine.

15. A method for reducing sulfides from a fluid stream comprising:
(g) providing a fluid stream; and
(h) contacting said sulfides in said fluid stream with least one secondary amine-formaldehyde adduct (SAFA) scavenger wherein said SAFA scavenger comprises less than about 40 wt % N-methyl secondary amines therein.

16. The method of claim 15, wherein said sulfides comprise one or more members selected from the group consisting of organic sulfides, mercaptans, thiols, COS, and H₂S.

17. The method of claim 15, wherein said sulfides are H₂S.

18. The method of claim 15, wherein said fluid stream is a hydrocarbon stream.

19. The method of claim 15, wherein said fluid stream is an aqueous stream.

20. The method of claim 15, wherein said secondary amine-formaldehyde adduct (SAFA) scavenger comprises less than about 20 wt % N-methyl secondary amines therein.

21. The method of claim 15, wherein said secondary amine-formaldehyde adduct (SAFA) scavenger comprises less than about 5 wt % N-methyl secondary amines therein.

22. The method of claim 15, wherein said secondary amine-formaldehyde adduct (SAFA) scavenger is added to said fluid stream in an amount ranging from about 10 to about 100,000 ppm by volume of said fluid stream.

23. The method of claim 15, wherein said secondary amine-formaldehyde adduct (SAFA) scavenger is added to said fluid stream in an amount ranging from about 100 to about 50,000 ppm by volume of said fluid stream.

24. The method of claim 15, wherein said secondary amine-formaldehyde adduct (SAFA) scavenger is added to said fluid stream in an amount ranging from about 600 to about 3,000 ppm by volume of said fluid stream.

25. Secondary amine-formaldehyde adduct (SAFA) scavengers comprising less than about 40 wt % N-methyl secondary amines therein.

26. The SAFA scavengers of claim 25, wherein said SAFA scavengers comprise a dibutylamine-formaldehyde adduct scavenger having the formula: 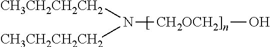
where n may be 1 to 100.

27. The SAFA scavengers of claim 25, wherein said SAFA scavengers comprise a dibutylamine-formaldehyde adduct scavenger having the formula: 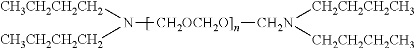
where n may be 1 to 100.

28. The SAFA scavengers of claim 25, wherein said SAFA scavengers comprise less than about 20 wt % N-methyl secondary amines therein.

29. The SAFA scavengers of claim 25, wherein said SAFA scavengers comprise less than about 5 wt % N-methyl secondary amines therein.
